(19) United States
(12) Patent Application Publication
(10) Pub. No.: US 2005/0038125 A1
(43) Pub. Date: Feb. 17, 2005

(54) Method for the treatment of arthritis and pain

(76) Inventors: Hobbe Friso Smit, Utrecht (NL); Sander Hougee, Wageningen (NL); John Sijben, Wageningen (NL)

Correspondence Address: YOUNG & THOMPSON, 745 SOUTH 23RD STREET, 2ND FLOOR, ARLINGTON, VA, 22202, US

(21) Appl. No.: 10/641,122

(22) Filed: Aug. 15, 2003

(51) Int. Cl. A61K 31/075

(52) U.S. Cl. 514/720

(57) ABSTRACT

The present invention relates to a method for the treatment and/or prevention of disorders with elevated PGE₂ and/or LTB₄ levels, comprising administering to a mammal an effective amount of pterostilbene component (PS component), a pharmaceutically acceptable salt of PS component or a precursor of PS component, wherein the PS component has the formula 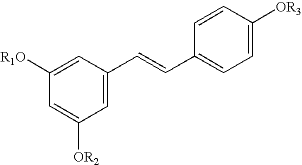
in which R1, R2 and R3 are independently selected from hydrogen, C₁₋₅₀ hydrocarbyl, C₁₋₅₀ substituted hydrocarbyl, C₁₋₅₀ heterohydrocarbyl, C₁₋₅₀ substituted heterohydrocarbyl; and wherein at least one of R1 and R2 is not hydrogen

### FIELD OF THE INVENTION

The present invention relates to a method for the treatment and/or prevention of disorders with elevated PGE₂ and/or LTB₄ levels in mammals, said method comprising the administration of a component capable of reducing PGE₂ and/or LTB₄ production.

### BACKGROUND OF THE INVENTION

Inflammatory conditions of the joint are often chronic joint diseases which afflict and disable, to varying degrees, millions of people worldwide. Arthritis is one of the most common disabling diseases. It has been estimated that about 10 percent of the Western population suffers from one or many forms of atbritis. There are over 75 types of different diseases of the joint, classified according to their specific symptoms and causes. Representatives of the more common disorders are rheumatoid arthritis and osteoarthritis.

Prostaglandins and leukotrienes are lipid mediators produced in a variety of inflammatory disease states, including inflammatory conditions of the joint. Both are products of metabolism of arachidonic acid. Cyclooxygenases (COX-1 and COX-2) are the enzymes that catalyze the conversion of arachidonic acid to prostaglandins. 5-Lipoxygenase (5-LO) catalyzes the conversion of arachidonic acid to leukotrienes.

Patients suffering from arthritis often have relatively high prostaglandin E2 (PGE₂) levels. PGE₂ induces inflammation and causes the sensation of pain. In addition PGE₂ is involved in deterioration of the cartilage.

A variety of biological effects is associated with leukotrienes, the products of lipoxygenase activity, and many are implicated as mediators in various disease states. Leukotriene B4 (LTB₄) and 5-hydroxyeicosatetraenoic acid (5-HETE) are potent chemotactic factors for inflammatory cells such as polymorphonuclear leukocytes. They are also found in the synovial fluid of patients with rheumatoid arthritis. Considerable efforts have been directed toward the control of leukotriene biosynthesis. Generally, research efforts have been directed toward the discovery of inhibitors of the 5-LO pathway and, in particular, 5-LO specific inhibitors.

Also in patients suffering from fibromyalgia and local myalgia increased levels of PGE₂ and LTB₄ have been found.

Hence, compositions capable of reducing or preventing PGE₂ production and/or LTB₄ production are highly desired.

### SUMMARY OF TEE INVENTION

The present inventors found that pterostilbene component (see below; PS component) is capable of effectively reducing PGE₂ production. Moreover, it was found that oral administration of PS component inhibits PGE₂ production in mice. Hence, the PS component can be advantageously used in a method for the treatment or prevention of disorders with elevated PGE₂ levels, such as arthritis, fibromyalgia and pain.

In addition it was surprisingly found that PS component effectively inhibits LTB₄ production in mice, making the PS component suitable for use in a method for preventing and/or treating diseases with elevated LTB₄ production, such as asthma, allergy, arthritis, psoriasis, fibromyalgia and inflammation.

The present invention also provides compositions, which can be advantageously used in a method for the treatment or prevention of disorders with elevated PGE₂ and/or LTB₄ levels, particularly inflammatory disorders of the joint. This composition comprises the present PS component and a constituent capable of stimulating proteoglycan synthesis, particularly constituents selected from the group consisting of glucosamine, chondroitin, collagen type II, S-adenosylmethionine, methylsulfonylmethane (MSM), hydroxyproline and mixtures thereof.

### DETAILED DESCRIPTION OF THE PREFFERED EMBODIMENTS

The present invention provides a method for the treatment or prevention of disorders with elevated PGE₂ and/or LTB₄ levels in a mammal, said method comprising administering to said mammal an effective amount of pterostilbene component (PS component), a pharmaceutically acceptable salt of said PS component or a precursor of said PS component. Preferably PS component is administered enterally.

The pterostilbene component has the formula 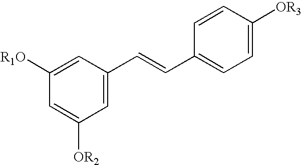
in which R1, R2 and R3 are independently selected from hydrogen, C₁₋₅₀ hydrocarbyl, C₁₋₅₀ so substituted hydrocarbyl, C₁₋₅₀ heterohydrocarbyl, C₁₋₅₀ substituted heterohydrocarbyl; and wherein at least one of R1 and R2 is not hydrogen. With the proviso that at least one of R1 and R2 is not hydrogen, preferably at least one of (and preferably both) R1 and R2 are independently selected from C₁₋₃₀ hydrocarbyl, C₁₋₃₀ substituted hydrocarbyl, C₁₋₃₀ heterohydrocarbyl, C₁₋₃₀ substituted heterohydrocarbyl and in particular at least one of (and preferably both) R1 and R2 are independently selected from C₁₋₃₀ hydrocarbyl, even more preferably at least one of R1 and R2 represent methyl, most preferably R1 and R2 represent methyl. Preferably, R3 represents hydrogen. Preferably the PS component is selected from the group consisting of 3,4′-dihydroxy-5-methoxystilbene, 4′-hydroxy-3,5-dimethoxystilbene and 5,4′-dihydroxy-3-methoxystilbene. The preferred PS component is 4′-hydroxy-3,5-dimethoxystilbene (i.e. pterostilbene).

The term “hydrocarbyl” denotes a group having a hydrocarbon or predominantly hydrocarbon character. Such groups include the following:

(1) Purely hydrocarbon groups; that is, aliphatic, (e.g., alkyl or alkenyl), alicyclic (e.g., cycloalkyl or cycloalkenyl), aromatic, aliphatic- and alicyclic-substituted aromatic, aromatic-substituted aliphatic and alicyclic groups, and the like, as well as cyclic groups wherein the ring is completed through another portion of the molecule (that is, any two indicated substituents may together form an alicyclic group). Such groups are known to those skilled in the art. Examples include methyl, ethyl, octyl, decyl, octadecyl, cyclohexyl, phenyl, etc.

(2) Substituted hydrocarbon groups; that is, groups containing non-hydrocarbon substituents which do not alter the predominantly hydrocarbon character of the group. Those skilled in the art will be aware of suitable substituents. Examples include hydroxy, nitro, cyano, alkoxy, acyl, etc.

(3) Hetero groups; that is, groups which, while predominantly hydrocarbon in character, contain atoms other than carbon in a chain or ring otherwise composed of carbon atoms. Suitable hetero atoms will be apparent to those skilled in the art and include, for example, nitrogen, oxygen and sulfur.

Precursors of the PS component are compounds that can be converted into PS component by gastrointestinal hydrolytic cleavage, such as for instance carbohydrate derivatives, carbonate derivatives, ester derivatives and ether derivatives. Preferably, the precursors of the PS component are selected from the group consisting of glycosides, rutinosides, glucuronoside, gentobioside and methyl ethers of PS component, more preferably glycosides of PS component.

The group consisting of pterostilbene component (PS component), a pharmaceutically acceptable salt of said PS component and a precursor of said PS component is hereinafter commonly referred to as “PS component”.

Sources of PS Component

The PS component used in the present method may be chemically manufactured (e.g. by partial methylation of trans- (Rimando et al, J. Agric. Food Chem (2002) 50: 3453-3457), biochemically produced (e.g. in fermentation processes), or be obtained from vegetable material, optionally followed by subsequent chemical modification. Subsequent chemical modification includes the preparation of precursors of PS component such as for instance glycosylation to produce glycosides of PS component or acylation to produce ester-group containing PS component.

The PS component is preferably isolated from plant material, particularly from the heartwood, wood and or bark of plants. Preferred plant material for obtaining the PS component are selected from the group consisting of Guibourtia plants, Pterocarpus plants, Vitis plants and Dracaena plant More preferably the plant material is selected from the group consisting of Guibourtia tessmanii, Pterocarpus santalinus, Pterocaxpus marsupium, Pterocarpus dalbergioides, Pterocarpus macrocarpus, Pterocarpus soyauxii, Pterocarpus tinctorius, Vitis vinefera, Dracaena cochinensis, Pterolobium hexapetalum and Dracaena loureiri, even more preferably selected from the group consisting of Guibourtia tessmanii, Pterocarpus santalinus, Pterocaxpus marsupiul, Vitis vinefera, and most preferably selected from the group consisting of Pterocarpus santalinus and Pterocarpus marsupium. As mentioned above

In a preferred embodiment, the present method comprises the administration of an effective amount of a plant isolate, said plant isolate containing at least 1 wt. % PS component, based on dry weight of the plant isolate, preferably at least 2.5 wt. %, even more preferably at least 5 wt. %, even more preferably at least 10 wt. %, most preferably at least 40 wt. %. Preferably the plant isolate is obtained from one or more of the above-mentioned plants. An isolate containing PS component may also comprise precursors of PS component such as for instance glycosides esters.

The term “isolate” as referred to in here, encompasses any fraction that can be obtained from a plant material by means of isolation techniques known in the art, e.g. extraction, distillation, squeezing etc. and that displays the desired functional properties described herein before. The term “extract” as used in the present invention refers to an isolate that has been obtained by means of solvent extraction.

The plant isolate is preferably prepared by solvent extraction, more preferably by extraction of the plant raw material with an non-aqueous solvent. According to an even more preferred embodiment, the plant isolate is prepared by an isolation process which includes at least a solvent extraction and at least a chromatographic separation technique.

Dosages of PS Component

The present method preferably comprises the administration of an effective amount of PS component. Preferably, a daily amount of between 0.01 and 250 mg PS component per kg metabolic weight of the mammal is administered to a mammal, more preferably between 0.1 and 100 mg/kg metabolic weight, even more preferably between 0.5 and 50 mg/kg metabolic weight, most preferably between 0.75 and 25 mg/kg metabolic weight The metabolic weight of a mammal is calculated with the following formula:
Metabolic weight=(body weight)^{0.75.}

For a human, the daily amount administered is preferably between 0.01 and 100 mg PS component per kg body weight, more preferably between 0.05 and 50 mg/kg body weight, even more preferably between 0.1 and 25 mg/kg body weight, most preferably between 0.2 and 20 mg/kg body weight.

In a further embodiment, the present method comprises the administration of a dosage comprising between 1 mg and 10 g PS component, more preferably between 2.5 mg and 1 gram, even more preferably between 10 mg and 250 mg, even more preferably between 15 and 200 mg. These dosages are particularly suitable for administration to humans.

Treatment

The present invention provides a method for reducing or preventing disorders with elevated PGE₂ and/or LTB₄ levels, particularly pain, inflammation, joint disorders, asthma, allergy, arthritis, fibromyalgia and psoriasis.

The PS component can advantageously be used in a method for the treatment or prevention of joint disorders selected from the group consisting of osteoarthritis, psoriatic arthritis, rheumatoid arthritis; for stimulating joint health; and is especially useful in a method for the treatment, repair or prevention of damage to the connective tissue.

Inflammatory conditions which can be advantageously treated or prevented with the present method are inflammatory condition resulting from strain, sprain, cartilage damage, trauma, orthopedic surgery, infection or other disease processes.

Also fibromyalgia, a chronic disorder characterized by widespread pain in the muscles, ligaments and tendons—the fibrous tissues in the body—and for which the exact cause is still unknown, can be advantageously treated with the present method.

The present method, being particularly aimed at the treatment, repair or prevention of damage to the connective tissue, may be manifested in the form of products, such as nutritional supplements, advertised to treat or prevent undesirable conditions of the joint and/or the treatment or prevention of symptoms thereof. Nutritional supplements labeled with terminology pointing to the above method are encompassed by the present method. Such terminology includes for example “joint health”, “mobility”, “cartilage plus”, “artri-cure”, “joint pain”, “flexibility”, “joint-repair”, “cartila™”, “osteobiflex™”, “joint comfort” or “joint-flex™”. The term “symptoms” includes pain, tenderness of the joints, swelling, stiffness, morning stiffness and loss of cartilage.

Administration

Preferably, the present method comprises the administration of PS component incorporated in a pharmaceutical preparation or nutritional supplement. This preparation preferably comprises the PS component and a pharmaceutically acceptable carrier. Packaged nutritional supplements and dietary products, which have been provided with labels that explicitly or implicitly direct the consumer towards the use of said supplement or product in accordance with one or more of the methods described above or below, are encompassed by the present invention.

In accordance with the present method the PS component is preferably administered enternally, more preferably orally. The PS component used in the present method cal be applied in any suitable form, such as meals, bars, pills (e.g. capsules, tablets or caplets), gels, biscuits and drinks.

According to a further preferred embodiment the PS component is provided in a unit dosage form. The term unit dosage form refers to a physically discrete unit suitable for unitary administration to human subjects and other mammals, wherein each unit contains a predetermined quantity of PS component and a pharmaceutically acceptable carrier.

The aforementioned unit dosage form is preferably ill a solid or semisolid form, more preferably in the form of an oral dosage unit, which term includes capsules, tablets, microparticles and microspheres.

The PS component used in the present method may be modified in environments of the upper intestinal tract and is therefore preferably orally administered in a unit dosage form that is coated with a substance that can withstand the enteric environment (an enteric coating) to prevent the decrease of its activity.

The solid or semisolid unit dosage form preferably has a weight between 0.1 and 30 grams, more preferably between 0.2 and 10 gram. When an oral dosage unit is used to provide the PS component, it preferably has a weight between 0.2 and 4 grams, even more preferably between 0.5 and 3 grams. In the present method a daily dosage of PS component can include one or more unit dosage forms, preferably the daily dosage consists of 1 to 6 unit dosage forms.

In a further aspect, the present invention provides a container that comprises multiple PS component containing dosages. The container bears a label, which indicates that the dosages should be ingested by a human suffering from pain, inflammation, joint disorders, asthma, allergy, arthritis, fibromyalgia or psoriasis, preferably by humans which aim to treat, prevent or repair damaged connective tissue and/or to restore cartilage function. The container preferably contains between 2 and 250 dosages, more preferably between 7 and 150 dosages, even more preferably between 25 and 100 dosages per container.

Combinations

The present inventors found that the present PS component can be advantageously used in a method for treatment or prevention of disorders with elevated PGE₂ and/or LTB₄ levels, particularly joint disorders. The present method can be further improved by co-administering with the PS component, constituents capable of stimulating proteoglycan synthesis.

Hence, the present invention also provides compositions suitable for the treatment or prevention of disorders with elevated PGE₂ and/or LTB₄ levels in a mammal (particularly joint disorders such as arthritis), comprising the PS component and a constituent selected from the group consisting of glucosamine, chondroitine, collagen type II, S-adenosylmethionine, methylsulfonylmethane (MSM) and hydroxyproline; preferably glucosamine.

In a further preferred embodiment, the present invention provides compositions suitable for the treatment or prevention of joint disorders such as arthritis, comprising the PS component and docosahexaenoic acid and/or eicosapentaenoic acid.

Also this invention encompasses the use of PS component as defined above for the preparation of a composition or medicament for the treatment and/or prevention of conditions or disorders with elevated PGE₂ and/or LTB₄, such as for example pain, inflammation, joint disorders, asthma, allergy, arthritis, fibromyalgia and psoriasis.

### EXAMPLES

### Example 1

### PGE₂ Production Inhibitor Effects of Pterostilbene, Silbinol™ and Resveratrol on Lipopolysaccharide (LPS) Stimulated hPBMCs

Peripheral Blood Mononuclear Cells (PBMCs) isolated from buffy coats of human blood from 3 healthy donors were stored in liquid nitrogen using standard procedures. For each experiment, PBMCs were thawed, transferred into a 50 ml centrifuge tube (BD Falcon, Erembodegem Aalst, Belgium) and cell culture medium (RPNI-1640 containing 25 mM HEPES and 2 mM L-glutamine (Life-Technologies, Merelbeke, Belgium) and further enriched with 10% heat inactivated fetal calf serum (^{hi}FCS), 120 U/ml penicillin/streptomycin and 1.2 mM sodium pyruvate) was added in a drop-wise fashion. Subsequently cells were centrifuged for 5 minutes at 1200 rpm (Sorvall RT7™). Cells were washed with cell culture medium (two times) and counted using a coulter counter (Beckman Coulter, Fullerton, Calif., USA). Parallel, a microscopic viability check using trypan blue is performed. PBMCs were pipetted into a 96-well flat bottom microtiterplate (BD Falcon, Brembodegem Aalst, Belgium), 1.5×10⁵ cells per well. Pterostilbene (Chromadex, Santa Anna, Calif., USA), Silbinol™ (Pterocarpus marsupium plant extract containing at least 5 wt. % pterostilbene based on dried weight; Sabinsa Corporation, Piscataway, USA) and resveratrol (Sigma, St. Louis, USA) were dissolved in dimethylsulfoxide (DMSO) and subsequently further diluted in cell culture medium and added to the cells in a concentration range (see below). DMSO concentration in the wells was 0.1%. Subsequently the cells were incubated for 1 hour at 37° C. in a humidified atmosphere containing 5% CO₂. After 1 hour, lipopolysaccharide (LPS, E. Coli B55:O55, 10 ng/mW) was added and the cells were incubated for 20 hours at 37° C. in a humidified atmosphere containing 5% CO₂. The supernatants were harvested 20 hours after incubation and stored at −80° C. until further analysis. Metabolic activity of the PBMCs with and without the presence of test compound was determined using the succinate dehydrogenase activity.

Concentrations of 3.12, 6.25, 12.5, 25 and 50 μg/ml of either Pterostilbene or Silbinol™ were tested to find the dose range of inhibitory activity (Table 1 and 2). Following the determination of the dose range with PGE₂ production inhibitory activity, concentrations of 0, 2, 4, 8, 16 and 32 μg/ml Silbinol™ (Table 3) and 0, 0.037, 0.11, 0.33, 1 and 3 μM pterostilbene (Table 4) were tested.

For comparative purposes, the effects 0, 0.33, 1, 3, 9 and 27 μM resveratrol on PGE₂ production by hPBMCs was determined (Table 5).

PGE₂ in the thawed supernatants was measured using a commercial enzyme immunoassay (Biotrak Amersham, Buckinghamshire, UK) according to the manufacturer's protocol 2.

TABLE 1
Effect of 0 to 50 μg/ml Pterostilbene on PGE₂ production (pg/ml) by
hPBMCs after LPS stimulation. PGE₂ values are the averages of
duplicate measurements.

| | Concentration Pterostilbene (μg/ml) | | | | | |
|---|---|---|---|---|---|---|
| | 0 | 3.12 | 6.25 | 12.5 | 25 | 50 |
| donor A (pg/ml) | 5865 | ND¹ | 28 | ND¹ | 63 | 584 |
| donor B (pg/ml) | 2937 | 117 | ND¹ | 5 | 69 | 222 |
| donor C (pg/ml) | 2343 | ND¹ | ND¹ | ND¹ | ND¹ | ND¹ |
| Mean (pg/ml) | 3715 | 39 | 9 | 2 | 44 | 269 |

¹Not detectable: the values do not reach the detection level

TABLE 2
Effect of 0 to 50 μg/ml PS component on PGE₂ production (pg/ml) by
hPBMCs after LPS stimulation PGE₂ values are the averages of
duplicate measurements.

| | Concentration Silbinol ™ (μg/ml) | | | | | |
|---|---|---|---|---|---|---|
| | 0 | 3.12 | 6.25 | 12.5 | 25 | 50 |
| donor A (pg/ml) | 3657 | 2278 | 2267 | 1329 | 868 | ND¹ |
| donor B (pg/ml) | 2881 | 1865 | 1511 | 1337 | 1040 | 590 |
| donor C (pg/ml) | 3193 | 2196 | 1771 | 1699 | 829 | 535 |
| Mean (pg/ml) | 3244 | 2113 | 1850 | 1455 | 912 | 375 |

¹Not detectable: the values do not reach the detection level

TABLE 3
Effect of 0 to 32 μg/ml Silbinol ™ on PGE₂ production (pg/ml) by
hPBMCs after LPS stimulation. PGE₂ values are the averages of
duplicate measurements.

| | Concentration Silbinol ™ (μg/ml) | | | | | |
|---|---|---|---|---|---|---|
| | 0 | 2 | 4 | 8 | 16 | 32 |
| donor A (pg/ml) | 5544 | 3133 | 2116 | 668 | 45 | 24 |
| donor B (pg/ml) | 4538 | 2590 | 2385 | 1666 | 1347 | 1528 |
| donor C (pg/ml) | 3586 | 231 | ND¹ | ND¹ | ND¹ | ND¹ |
| Mean (pg/ml) | 4556 | 1985 | 1500 | 778 | 464 | 517 |

¹Not detectable: the values do not reach the detection level

TABLE 4
Effect of 0 to 3 μM Pterostilbene on PGE₂ production (pg/ml) by
hPBMCs after LPS stimulation. PGE₂ values are the averages of
duplicate measurements.

| | Concentration Pterostilbene (μM) | | | | | |
|---|---|---|---|---|---|---|
| | 0 | 0.037 | 0.11 | 0.33 | 1 | 3 |
| donor A (pg/ml) | 5076 | 5957 | 5305 | 2786 | 2067 | 280 |
| donor B (pg/ml) | 5898 | 7788 | 6930 | 4782 | 3570 | 1794 |
| donor C (pg/ml) | 2143 | 2574 | 1248 | 199 | ND¹ | ND¹ |
| Mean (pg/ml) | 4372 | 5434 | 4494 | 2589 | 1879 | 691 |

¹Not detectable: the values do not reach the detection level

TABLE 5
Effect of 0 to 27 μM Resveratrol on PGE₂ production (pg/ml) by
hPBMCs after LPS stimulation. PGE₂ values are the averages of
duplicate measurements.

| | Concentration Resveratrol (μM) in culture | | | | | |
|---|---|---|---|---|---|---|
| | 0 | 0.33 | 1 | 3 | 9 | 27 |
| donor A (pg/ml) | 6537 | 8380 | 6835 | 3120 | 1213 | 913 |
| donor B (pg/ml) | 6035 | 8060 | 7369 | 5449 | 2936 | 2355 |
| donor C (pg/ml) | 3115 | 3710 | 2597 | 178 | ND¹ | ND¹ |
| Mean (pg/ml) | 5229 | 6717 | 5601 | 2916 | 1383 | 1090 |

¹Not detectable: the values do not reach the detection level

From the above it is apparent that Silbinol™, Pterostilbene and Resveratrol inhibit PGE₂ production by hPBMCs. In the “lower” dose range (see Table 4) pterostilbene inhibited PGE₂ production by hPBMCs in a dose dependent manner. The metabolic activity of hPBMCs was found to decrease, however to a much lesser extent. The estimated IC50 value of pterostilbene was 0.83 μM. Resveratrol also inhibits PGE₂ production by hPBMCs after LPS stimulation (Table 5), with an estimated IC50 of 4.7 μM.

The above experiment is indicative for the suitability of pterostilbene component and pterostilbene (enriched) extracts for use in the present method. These results are also indicative for the enhanced potency effects of pterostilbene and extracts comprising pterostilbene as compared to resveratrol.

### Example 2

### Effects of Silbinol™ and Ibuprofen™ on LPS Stimulated PGE₂ Production by Mouse Whole Blood

Murine blood (C57/b16 male) was collected in heparinized collection tubes. The blood was diluted 0.5 times with cell culture medium without serum. 150 μl diluted blood was transferred into a 96-well microtiterplate. Silbinol™ was dissolved in DMSO and subsequently further diluted before adding to the cells in a concentration range of 0-25-50 μg/ml. Total amount of DMSO in the well was 0.1% DMSO. After 1 hour of preincubation of the mixture of blood cells and Silbinol™ or blood cells and 100 μM Ibuprofen™ at 37° C. in a humidified atmosphere containing 5% CO₂, 1 μg/ml LPS was added and incubated for another 20 hours. Plates were centrifuged (5 minutes, 1200 rpm, SorvallRT7™) and supernatants are harvested and stored at −80° C. for further analysis.

PGE₂ in the thawed supernatants was determined using the commercial enzyme immunoassay of Biotrak™ (Biotrak Amersham, Buckinghamshire, UK) according to the manufacturer's protocol 2. The results are depicted in Table 6.

TABLE 6
Effects of Silbinol ™ and ibuprofen ™ on LPS stimulated PGE₂
production in mouse whole blood. PGE₂ values are the averages of
duplicate measurements.

| | | | | |
|---|---|---|---|---|
| Concentration Ibuprofen (μM) | | | | 100 |
| Concentration Silbinol ™ (μg/ml) | 0 | 25 | 50 | |
| PGE₂ production (pg/ml) | 19154 | 7047 | 4487 | 1709 |

Silbinol™ and Ibuprofen™ inhibit PGE₂ production in mouse whole blood. These data are indicative of the advantageous use of a pterostilbene enriched plant extract in the present method.

### Example 3

### Enteral Administration of Silbinol™ to Mice and Subsequent Ex Vivo determinattion of PGE2 and LTB₄ it Whole Blood

Male C57/b16 mice, age 12 weeks, were fed standard rodent diet ad libitum and water ad libitum. Silbinol™ was suspended in normal tap water fresh every day. The group receiving Silbinol™ (n=was supplemented with a daily dosage of 50 mg Silbinol™ per kg body weight through gavage (200 μl) for 10 days. The placebo group (n=7) received normal tap water through gavage. At day 8, 6 μl of a 30 mg/ml zymosan solution (1% Saccharomyces cerevisiae, Sigma, St. Louis, USA) solution was injected intra-acularly into the right knee joint. Two days later (day 10) the animals were anaestlesized by ether. Blood was collected into heparinized collection tubes and diluted 0.5 time with cell culture medium without serum. 150 μl blood was transferred into a 96 well plate and 50 μl cell culture medium [RPMI 1640 (2 mM L-Glut, 25 mM Hepes), 1 mM Na-Pyruvaat, 100 U/ml Pen/strep, (all Gibco, Carlsbad, Calif.)) was added.

Supernatants were analyzed for PGE₂ concentration using the Biotak™ PGE₂ assay (Biotrak Amersham Buckinghamshire, UK) and for LTB₄, concentration with a commercially available assay of RnD systems™ (RnD systems, Abingdon, United Kingdom).

Average ex vivo PGE₂ production in the Silbinol™ supplemented mice was approximately 44% lower than in the mice receiving placebo (P<0.001). Average a viva LTB₄ production was approximately 36% lower in the Silbinol™ treated animals compared with the placebo controls.

These results are indicative for the advantageous use of PS component and PS component containing isolate in the present method.

### Example 4

### Composition for the Treatment and/or Prevention of Damage to the Human Cartilage

A container comprising unit dosages, said container bearing a label indicating that the unit dosages should be orally ingested by subjects wishing to reduce or prevent damage to the cartilage, said unit dosage comprising:
450 mg Silbinol™ (providing about 22.5 mg pterostilbene)
500 mg glucosamine
600 mg tablet excipient

1. Method for the treatment or prevention of disorders with elevated PGE₂ and/or LTB₄ levels in a mammal, said method comprising enterally administering to said mammal an effective amount of pterostilbene component (PS component), a pharmaceutically acceptable salt of said PS component or a precursor of said PS component, wherein the pterostilbene component has the formula 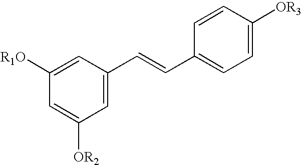
in which R1, R2 and R3 are independently selected from hydrogen, C₁₋₅₀ hydrocarbyl, C₁₋₅₀ substituted hydrocarbyl, C₁₋₅₀ heterohydrocarbyl, C₁₋₅₀ substituted heterohydrocarbyl and at least one of R1 and R2 is not hydrogen.

2. Method according to claim 1, wherein R1 and R2 represent C₁₋₁₀hydrocarbyl

3. Method according to claim 1, wherein R3 represents hydrogen

4. Method according to claim 1, for the treatment or prevention of joint disorders.

5. Method according to claim 1, for the treatment or prevention of inflammation.

6. Method according to claim 1, for the treatment or prevention of pain.

7. Method according to claim 4, for the treatment or prevention of rheumatoid arthritis or osteoarthritis.

8. Method according to claim 1, for the treatment or prevention of fibromyalgia.

9. Method according to claim 1, wherein the pterostibene component is obtained from plant material.

10. Method according to claim 1, wherein the method comprises the administration of a plant isolate which contains at least 1 wt. % pterostilbene component based on the dry weight of the isolate.

11. Method according to claim 1, comprising the daily administration of between 0.01 and 250 mg pterostilbene component per kg metabolic weight.

12. Method according to claim 1, comprising the administration of a dosage containing between 2.5 mg and 1 g pterostilbene component.

13. Method according to claim 1 comprising the coadministration of a constituent selected from the group consisting of glucosamine, chondroitin, collage type II, S-adenosylmethionine, methylsulfonylmethane (MSM), hydroxyproline and mixtures thereof.

14. A composition suitable for the treatment and or prevention of disorders with elevated PGE₂ and/or LTB₄ levels in a mammal comprising:
a, between 1 mg and 10 g of pterostilbene component, a pharmaceutically acceptable salt of said PS component or a precursor of said PS component as defined in claim 1; and
b. a constituent selected from the group consisting of glucose, chondroitin, collagen type II, S-adenosylmethionine, methylsulfonylmethane (MSM), hydroxyproline and mixtures thereof.

15. Composition according to claim 14, comprising glucosamine.

16. A container comprising oral dosages and bearing a label indicating that the contents should be orally ingested by a human suffering from pain, inflammation, joint disorders, asthma, allergy, arthritis, fibromyalgia or psoriasis, said dosages containing between 1 mg and 10 g PS component each.

17. Container according to claim 16 with a label indicating that the contents should be orally ingested by a human desiring to treat, repair or prevent damage to the connective tissue and/or to restore cartilage function.

18. Container according to claim 16, comprising between 7 and 150 dosages.
